(19) United States
(12) Patent Application Publication
(10) Pub. No.: US 2023/0139018 A1
(43) Pub. Date: May 4, 2023

(54) SYSTEM AND METHOD FOR PRE-MEDICAL MONITORING AND EFFICIENT PATIENT NAVIGATION

(71) Applicant: Epiphany Innovations, LLC, Oak Ridge, TN (US)

(72) Inventors: Franklin Merritt, Panama City Beach, FL (US); Robert Eby, Oak Ridge, TN (US); William Eby, Oak Ridge, TN (US)

(73) Assignee: Epiphany Innovations, LLC, Oak Ridge, TN (US)

(21) Appl. No.: 17/817,121

(22) Filed: Aug. 3, 2022

Related U.S. Application Data
(60) Provisional application No. 63/274,072, filed on Nov. 1, 2021.

(51) Int. Cl. G16H 40/20; G16H 50/20

(52) U.S. Cl. CPC G16H 40/20; G16H 50/20

(57) ABSTRACT

A technology-enabled data collection and navigation service for monitoring an individual during their day-to-day activities outside of a healthcare system or before the individual has or is aware of a need of the healthcare system, collecting defined data related to that individual during those day-to-day activities, and, when appropriate to do so, efficiently navigating that individual into and through the healthcare system for the purpose of receiving appropriate medical care and, more specifically, navigating that individual to the location and medical provider that is most appropriate for providing the medical care needed based on the individual's medical need and the defined data collected by the system.

### CROSS-REFERENCE TO RELATED APPLICATION

This application claims the benefit of U.S. Provisional Application No. 63/274,072 filed Nov. 1, 2022, and entitled SYSTEM AND METHOD FOR PRE-MEDICAL MONITORING AND EFFICIENT PATIENT NAVIGATION, which is incorporated herein by reference in its entirety.

### FIELD

This application relates to a technology-enabled data collection and navigation service for monitoring an individual during their day-to-day activities outside of a healthcare system or before the individual has or is aware of a need of the healthcare system, collecting defined data related to that individual during those day-to-day activities, and, when appropriate to do so, efficiently navigating that individual into and through the healthcare system for the purpose of receiving appropriate medical care and, more specifically, navigating that individual to the location and medical provider that is most appropriate for providing the medical care needed based on the individual's medical need and the defined data collected by the system.

### BACKGROUND OF THE INVENTION

Modern day healthcare systems throughout the world, including particularly in America, face a number of challenges, which often result in high costs, delays, wasted resources, unnecessary exposures, and poor patient outcomes.

One primary issue that patients and healthcare systems face is simply the task of correctly identifying the medical need of a given patient and then correctly navigating that patient to the best and most appropriate medical provider. Most individuals that are outside of the medical profession lack sufficient medical knowledge to accurately diagnose their own or someone else's medical condition when faced with a given set of symptoms. Critically, they might be unable to distinguish between symptoms that are indicative of an emergency and that should be treated right away and those that are minor and require little, if any, medical treatment. For example, an abnormally rapid heart rate might, in some cases, be indicative of or caused by a serious and life threatening events such as; cardiac arrhythmias (e.g., atrial fibrillation, supra ventricular tachycardia or ventricular tachycardia), heart attack or heart failure, medication toxicity, thyroid storm, or “silent” internal bleeding, etc., where emergency medical treatment is needed. However, in other cases, a racing heartrate might be indicative of a less serious medical condition, such as strenuous exercise, stress/anxiety, or over-caffeination, where an emergency room visit is not required. As a result of the high stakes of this uncertainty, the default response is often to “play it safe” by utilizing the emergency room (ER) in all cases. This results in the overcrowding of ERs with patients that, frequently, do not require emergency care. On average, ERs are typically overcrowded with 25% or more cases being non-emergency cases. This overcrowding not only places a strain on ER staff and resources, but it also leads to crowds in the waiting rooms where persons are exposed to infectious diseases, increases the wait time for actual emergencies, and also delays care for non-emergencies since subsequent follow-up visits to primary care physicians or a specialist (e.g., a cardiologist) is frequently required.

Next, as medicine advances and becomes more specialized, including specializing hospitals and ERs, specialized practice groups, specialists within given practice groups, the complexity of the healthcare system as a whole also increases. While specialization and improvement in the healthcare system has the potential to provide better patient outcomes, the increased complexity of the healthcare system has also resulted in more frequent instances of “misnavigation” or “mislocating” of patients within that system. Once a patient has entered the healthcare system, they are “misnavigated” or “mislocated” when they seek treatment for their medical condition with an improper or less than ideal medical provider or medical facility.

In today's healthcare system, not all ERs or medical centers have the same equipment, staff, or capabilities. For example, some ERs are “trauma centers,” which are equipped and staffed to provide care for patients suffering from major traumatic injuries (e.g., injuries resulting from falls, motor vehicle accidents, gunshots, etc.). Additionally, within the trauma center category, facilities that are equipped and staffed to provide the most advanced and comprehensive care are classified as “Level I” trauma centers, whereas a “Level V” trauma center is equipped and staffed to provide only basic levels of treatment. In another example, certain medical centers are classified as “pediatric” medical centers that specialize in treating children, while others are classified as “stroke centers” that specialize in addressing strokes. Next, there is specialization even within specialty practice groups within the same organization. For example, a patient with a sprained ankle might be required to request an appointment with an orthopedist that specializes in ankles because she cannot be most effectively and efficiently treated by an orthopedist in that same group that specializes in knees.

As a result of this increased complexity and specialization within the healthcare system, the layperson often has no idea which facility or medical provider is appropriate for their condition. This frequently results in patients becoming lost within the healthcare system and then somewhat blindly finding their way to the appropriate medical provider, which often occurs only after needlessly spending significant amounts of time and money. Often, in order to locate the correct facility or medical provider, one or more trips to other facilities and other providers is required. Once there, that medical facility or medical provider acts as a navigator or traffic controller to then guide the patient to another facility or another medical provider to identify and treat the medical condition the patient faces. This scenario is akin to a person becoming lost in the woods and hoping to find a compass nailed to one of the trees that will assist them in finding their way through the woods. Unfortunately, in many cases, the time and money wasted in this process frequently results in the patient's medical condition worsening and reduces the resources that the patient has available to them to commit to the treatment of their condition. The cost to the patient and insurance company continues to rise exponentially as each of these mislocating visits mount. Overall, the current meandering pathway through the healthcare system is inefficient and frequently results in wasted resources and poor patient outcomes.

Another issue that today's healthcare systems face is underutilization, where a patient has an actual and, sometimes emergency, need of medical services but where those services are not sought or are not provided. This might occur, for example, in cases where a person is experiencing an asymptotic medical condition, illness, etc. and are not treated or are treated too late. As an example, around 15% of cardiac arrhythmia called atrial fibrillation are “silent” or asymptomatic with the individual being unaware of the condition which increases the individual's risk of stroke, heart failure, heart attack and other morbidities. The longer this conditions continues unnoticed, the higher the risks become. As another example, a “silent heart attack” occurs when the patient has a heart attack but does not experience any of the common symptoms (e.g., chest pain, shortness of breath, etc.). Nevertheless, having a silent heart attack places the patient at greater risk of having another heart attack later on, which could be deadly, and increases their risk of complications such as heart failure. The underuse of medical care is common and is responsible for substantial suffering, worsening of conditions, and loss of life worldwide.

Lastly, patients usually have very limited interactions with the learned medical professionals that care for them and that help them in making long-term decisions about their health. These interactions are often limited to just a few minutes, where the medical professional might review labs or vitals readings (e.g., blood pressure, heart rate, etc.) taken the day of the exam. However, this single, limited interaction frequently does not reveal the whole picture of the patient. One reason is that the timeframe is simply too short. A typical yearly doctor's appointment lasts approximately 30 minutes and the vital signs and physical exam portion of the appointment takes usually less than 5 minutes, but there are over 500,000 minutes in a year. Therefore, the period of observation occurring during these annual visits (if an annual visit even occurs) often accounts for less than 0.001% of the year. Next, certain warning signs are not easily measured, may not manifest in a short annual visit, or are not “medical” in nature. For example, slurred speech or impaired cognition (i.e., trouble remembering, learning new things, concentrating, etc.), paroxysmal atrial fibrillation (PAF), are important indicators of serious medical conditions that may not be easily measured or observed in a short physical examination. Similarly, weaving while driving a vehicle, trembling hands, and changing sleep patterns are not typical “medical” observations that a medical professional can make, but are indicators of possible underlying health issues. As a result, many obvious medical issues that would be quickly identified and addressed by medical professionals, if they were present to notice the associated symptoms or factors, are overlooked or missed entirely because the symptoms or factors that those medical professionals would use to identify those issues are not apparent during their limited interactions with patients.

What is needed, therefore, is a system and method for monitoring and gathering defined data related to individuals outside of the healthcare system in order to assist them in determining whether and when they are in need of medical services. Importantly, this monitoring and data gathering is “pre-medical” or occurring before the user enters the healthcare system. Additionally, in order to better represent the individual, the defined data gathered should be collected over a long period of time and should not be limited strictly to “medical” observations, but should include other non-medical observations as well. Lastly, once the users decide to enter the healthcare system, a system and method is also needed to efficiently guide them to the medical resources that are most appropriate for providing the medical care needed based on each individual's medical need and the defined data collected about that individual before they entered the healthcare system.

### Notes on Construction

The use of the terms “a”, “an”, “the” and similar terms in the context of describing the invention are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. The terms “substantially”, “generally” and other words of degree are relative modifiers intended to indicate permissible variation from the characteristic so modified. The use of such terms in describing a physical or functional characteristic of the invention is not intended to limit such characteristic to the absolute value which the term modifies, but rather to provide an approximation of the value of such physical or functional characteristic.

Terms concerning attachments, coupling and the like, such as “attached”, “connected” and “interconnected”, refer to a relationship wherein structures are secured or attached to one another either directly or indirectly through intervening structures, as well as both moveable and rigid attachments or relationships. The terms “operatively attached” and “operatively connected” describe such an attachment, coupling or connection that allows the pertinent structures to operate as intended by virtue of that relationship.

The use of any and all examples or exemplary language (e.g., “such as” and “preferably”) herein is intended merely to better illuminate the invention and the preferred embodiments thereof, and not to place a limitation on the scope of the invention. Nothing in the specification should be construed as indicating any element as essential to the practice of the invention unless so stated with specificity.

The term “defined data” means data received from or captured by a user technology device (e.g., cell phone, computer, smart wearable, or virtual assistant like Alexa). Preferably, defined data is transmitted directly or indirectly to the pre-medical monitoring and efficient patient navigation system of the present invention. Defined data includes traditional physiologic vital signs such as heart rate, breathing rate, blood pressure, oxygen saturation, and body temperature. Defined data may also include other or newly-created physiologic parameters, which are not conventionally considered “medical” in nature, and that may be used in evaluating users, including the neuromuscular and sensory aspects of speech patterns, gait patterns, sleep patterns (i.e., diurnal and nocturnal patterns), extremity movement patterns, patterns of user interface with technology (e.g., search inquiries, interactions with social media and gaming applications, texting, typing patterns, etc.).

The term “technology devices” is used and defined broadly to include any type of electronic device that may be nearby, interacted with, or carried by users. This interaction preferably occurs during at least a portion of the user's day. Even more preferably, this interaction occurs during the entire day and throughout the year. For example, without limitation, technology devices can include one or more the following: cell phones, wearables, computers, “smart” home devices (e.g., smart thermostat, smart speakers, smart watches, smart doorbells, etc.), vehicles, and the like.

The term “medical resources” means healthcare facilities, healthcare supplies or equipment, and/or healthcare providers.

The term “outside of” and the like, when used to refer to a user's involvement with the healthcare system or medical resources, means that the user is without (i.e., outside of) the medical system's active monitoring/observation, consultation, consideration, or advice for a specific medical issue, condition, symptom, etc. For example, a person that suspects they are having a heart attack or stroke is no longer “outside of” the healthcare system once they call 9-1-1 and are speaking with a dispatcher or travel to an emergency room and consult with a doctor. However, once that person ends the call with 9-1-1 or leaves the emergency room, such that they are no longer being monitored, considered, advised, etc. by a healthcare provider, they are outside of the healthcare system. In another example, a person is no longer “outside of” the healthcare system if they are provided with a take-home heart monitor that will enable the healthcare system to continue monitoring them for possible heart-related conditions or that will alert them leading up to or during a heart-related medical event (e.g., a heart attack). However, that same person is considered outside of the healthcare system if that heart monitor stops functioning such that the heart monitor would no longer observe the person or issue an alarm for a heart-related condition. Additionally, it is common for the healthcare system to focus on treating one medical condition and to miss another, unrelated medical condition. In those cases, with respect to the untreated and unaddressed medical condition, a person is “outside of” the healthcare system. For example, a person that calls 9-1-1 or travels to an emergency room for a laceration or broken arm may be “outside of” the healthcare system with respect to an impending heart attack or stroke for which the healthcare system is not actively monitoring/observing, consulting, considering, or advising the person on. Conversely, a user is “inside of,” “within,” or has “entered” the healthcare system for a particular medical issue, condition, symptom, etc., when they are receive active monitoring/observation, consultation, consideration, or advice for a specific medical issue, condition, symptom, etc. Thus, in certain cases, a user might be “within” the healthcare system with respect to one medical issue but not “within” the healthcare system with respect to another medical issue.

The term “pre-medical” means before a user has entered or is within the healthcare system.

The term “mis-utilized”, when used in describing medical resources, means medical resources that are overused, underused, or are misdirected. An overusage of medical resources occurs when more medical resources are used for identifying, diagnosing, treating, etc., a particular medical issue, condition, symptom, etc., than are necessary. For example, an overusage of medical resources occurs when a computed tomography (CT) scan is utilized in cases where a single X-ray or physical examination could be performed and achieve substantially the same result. This is an overusage of medical resources because of the higher monetary cost and radiation associated with the CT scan compared to the X-ray or physical examination. Conversely, an underusage of medical resources occurs when an insufficient amount of medical resources are used for identifying, diagnosing, treating, etc. a particular medical issue, condition, symptom, etc. For example, an underusage of medical resources occurs when a person does not seek medical treatment for a condition that should be treated by a medical provider. Lastly, medical resources are misdirected when an incorrect or less than ideal medical resource is utilized in identifying, diagnosing, treating, etc. a particular medical issue, condition, symptom, etc.

### BRIEF DESCRIPTION OF THE DRAWINGS

The presently preferred embodiment of the invention is illustrated in the accompanying drawings, in which like reference numerals represent like parts throughout, and wherein:

FIG. 1 is a schematic diagram depicting a pre-medical monitoring and efficient patient navigation system according to an embodiment of the present invention;

FIG. 2 is a flowchart illustrating a process of using the pre-medical monitoring and efficient patient navigation system of FIG. 1;

FIG. 3 depicts a mobile device issuing an alert and presenting options in response to a user being physically located near an emergency room;

FIG. 4 depicts a mobile device issuing an alert and presenting options in response to abnormal vital signs being sensed by a user's smart watch; and

FIG. 5 depicts a mobile device issuing an alert and presenting options in response to a user performing an Internet search on the mobile device.

### DESCRIPTION OF THE PREFERRED EMBODIMENT OF THE INVENTION

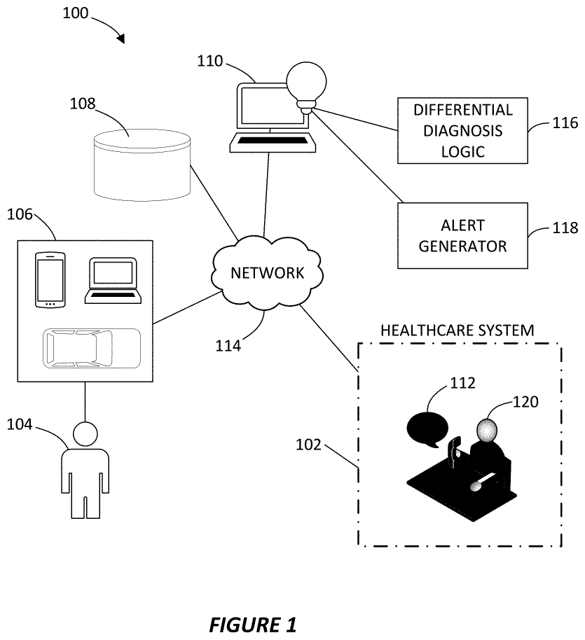

With reference now to FIG. 1, there is shown a pre-medical monitoring and efficient patient navigation system 100 for reducing instances of mis-utilization of medical resources within a healthcare system 102 and for efficiently navigating a user 104 to medical resources that are most appropriate for providing medical care required by the user. In general, the system 100 includes one or more technology devices 106, a data repository 108, a first triage system 110 located outside of the healthcare system 102, and a second triage system (represented here by question mark 112) located within the healthcare system, wherein some or all of these components are interconnected and communicate via a communications network 114. Each of these components are discussed in greater detail below.

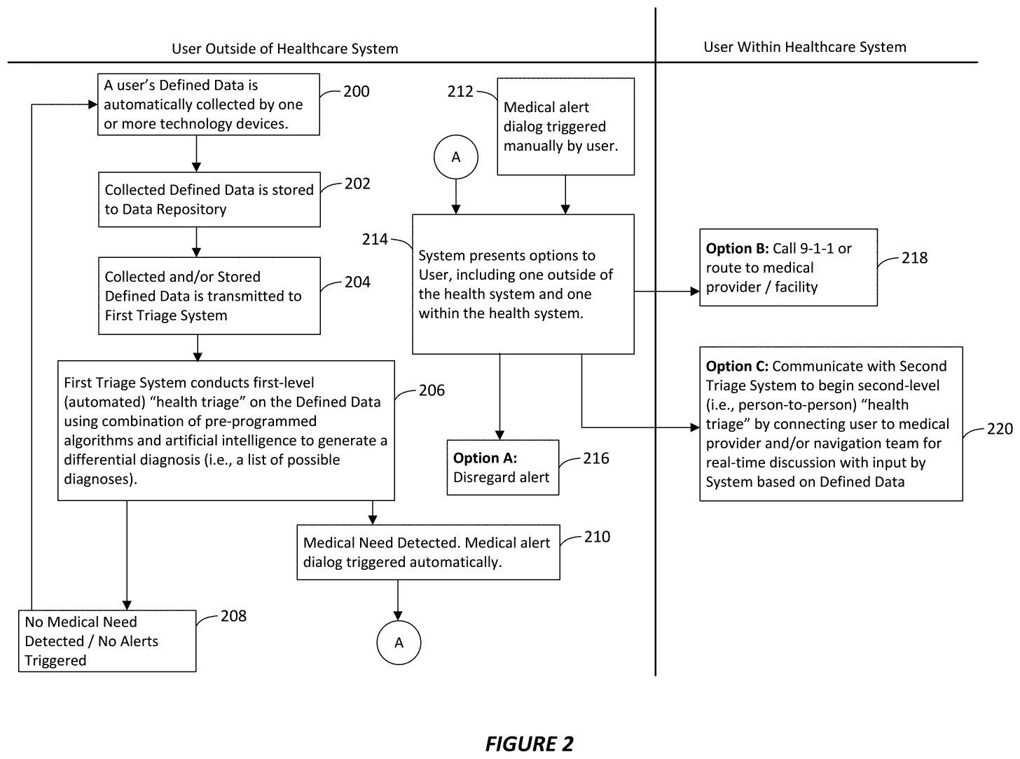

With continued reference to FIG. 1 and with further reference to FIG. 2, preferably, when using the presently-described system 100 and before a user interacts with the healthcare system 102, users of the system are monitored and various types of defined data related to the user are automatically collected by technology devices 106 (Step 200). In the past, in order to observe patients while they were outside of the healthcare facility (but inside a healthcare system), certain healthcare systems enabled monitoring of a patient's vital signs using a “hospital at home” device for a short period of time (e.g., 24 hours). For example, a Holter monitor was often used to look for cardiac arrhythmias. However, these devices were often limited in number and were bulky and hard to wear, which limited the amount of time the person could or would reliably wear the device. Additionally, these devices only monitored vital signs and were unable to monitor other physiological parameters like gait, speech, sleep patterns, extremity movement. The current system 100 leverages the widespread adoption and nearly continuous interaction with technology devices 106. Each of the technology devices 106 includes at least one data input receiver for receiving one or more types of defined data pertaining to the user 104. For example, in the case of a mobile phone, the device might include a speaker paired with voice recognition capabilities that can be used to record and recognize speech as well as changes in speech patterns or tone. The mobile phone might also be equipped with local or global position systems or components thereof, including accelerometers and GPS receivers, to detect a user's location and movement speed and direction. The mobile phone might further include a thumbprint sensor and/or other sensors that can detect temperature, heart rate, sweat production, hand steadiness, sleep patterns, etc. In short, the technology devices 106 of the system 100 of the present disclosure are capable of interfacing with and continually or almost continually tracking users' behavior and physiologic patterns and metrics.

In preferred embodiments, at least a portion of the defined data collected by the technology devices 106 is collected prior to the user entering (i.e., interacting with) a healthcare system 102. Additionally, this pre-medical defined data is collected passively (i.e., automatically and without requiring input from the user or without requiring the user to take any specific action) by the device 106 as the user carries out his or her daily activities. However, the system 100 is also designed to enable the user to voluntarily or actively provide other defined data, including before and after the user has entered the healthcare system 102, by inputting or providing defined data to the device 106. For example, the system 100 is configured to collect defined data that the user inputs into the device 106, such as Internet browser search strings and voice commands, and to use that information in making an assessment of the user. This defined data is stored in the data repository 108, which may include local storage on the device 106, remote storage on another device (e.g., data center, cloud storage, external drive), or both.

While not necessary, a data history is preferably generated by the wearer, including during an initialization stage of the system 100. This data history is collected by devices 106 and is then automatically stored in the data repository 108 (Step 202). The defined data is preferably associated with the user 104 to provide a user profile associated with that user, which is used to establish baselines for that user. Additionally or alternatively, the system 100 may be provided with a “factory” baseline for certain defined data. For example, certain ranges of vital signs might be preset based on medical standards and these baselines could be manually customized to the user and then automatically further adjusted by the system 100 as the system gathers more defined data about the user. For example, a system 100 might be preprogrammed with a default “normal” average resting heart rate of 75 beats per minute for all users. However, once the system 100 is initialized by a 50-year old male, this resting heart rate might be reduced to 70 beats per minute based on certain medical guidelines for that type of user, based on their age and gender. As the system 100 collects additional defined data about this specific, healthy user, the resting heart rate is further reduced to 63 beats per minute based on long-term monitoring. Similar manual and automatic adjustments may also be made for other defined data relevant to the user. These baselines assist the system 100 in determining what is “normal” and what is “abnormal” for that particular user. In addition to tracking and considering changes in a user's vital statistics, the system 100 will also review, track, and consider other non-medical defined data related to the user, including acute changes in a user's gait, speech (e.g., unusually slurred or slowed speech), sleep patterns (e.g., sleep hygiene, sleep apnea and insomnia), daily travel patterns, extremity movement, unusual mobile phone usage habits (e.g., unusual times of day), and the like to construct differential diagnoses, which might include altered mental status, stroke or transient ischemic attack (“TIA” or sometimes called a “mini stroke”), medication misuse or overdose, substance intoxication, psychosis, etc.

Some or all of the collected and stored defined data is automatically transmitted to the first triage system 110 (Step 204), which may be located locally on the technology devices 106 or may be located elsewhere and in remote communication with the technology devices via communications network 114. The first triage system 110 automatically conducts an analysis of the data provided to it. This analysis includes a first-level “health triage” or medical analysis, where the defined data may be cross-referenced with other data, including defined data from the user as well as information pertaining to potential medical conditions, to generate a differential diagnosis (i.e., a list of possible diagnoses) that might be affecting the user (Step 206). This differential diagnosis is created, at least in part, using a differential diagnosis logic 116 of the first triage system 110, which is preferably comprised of pre-programmed algorithms and artificial intelligence. This differential diagnosis is intended to assist the user and to also assist medical providers, to identify possible medical conditions that are impacting the user.

If the system 100 does not identify any medical condition, the analysis process is repeated and the system continues to monitor the user by collecting and analyzing defined data and other data (Step 208). The system 100 also preferably includes an alert generator 118 that is configured to generate an alert in response to an alert trigger. In preferred embodiments, one alert trigger is the differential diagnosis logic 116 identifying a possible medical condition. If the system 100 identifies a possible medical condition that might be affecting the user, the first triage system 110 issues an alert to at least one of the one or more technology devices 106 for review by the user 104 (Step 210), wherein the alert includes information related to the possible medical condition and one or more options to enter the healthcare system 102 for the purpose of obtaining medical treatment for the possible medical condition. In certain preferred embodiments, another alert trigger is the differential diagnosis logic 116 identifying that the one of the one or more technology devices 106 is located within a specified proximity of or is navigating towards (e.g., entering the parking lot of an emergency room or within a specified radius, set initially by the user, from an emergency room) a first medical resource having first qualifications. Preferably, the above-described review of data by the differential diagnosis logic 116 occurs automatically, without requiring input from the user, and before the user interacts with the healthcare system 110. However, the user 104 can also manually request an assessment on demand in the event that they recognize or suspect a medical need (Step 212).

Once an alert is triggered, the system 100 provides options that the user may take to address the condition (Step 214). These options are provided to the user 104 via one or more of the devices 106 with which the user interacts. For example, a push notification may be provided on the user's mobile phone, smart watch, or in a heads-up display or dashboard display in a vehicle. The system 100 is preferably configured to provide several options for responding to the alert. The user may respond or dismiss the alert by physically interacting with the device (e.g., via tapping, swiping, etc.). Additionally or alternatively, if the devices 106 are equipped with a speaker and microphone, the alert may be an audible alert and the options may be presented audibly. In that case, the user may also respond to the alert and presentation of options through verbal commands.

A first option (Option A) that is preferably presented by the system 100 is to simply disregard the alert (Step 216) and take no further action. The user would need to reactivate the triggering device to provide access for future alarms.

Next, the user is also preferably given the option (Option B) to immediately enter the healthcare system 102 (Step 218) by contacting 9-1-1 or by being routed to medical provider/medical facility. In cases where the user is navigating towards a first medical resource (e.g., a doctor, hospital, clinic, etc.), the differential diagnosis logic 116 will compare the possible medical condition identified to the qualifications of that first medical resource to determine if that medical resource is appropriate for addressing the medical condition. For example, if the possible medical condition is a stroke and the user is navigating towards a hospital, the differential diagnosis logic 116 will determine if that hospital is qualified to treat strokes. If the medical resource is appropriate (i.e., the hospital is a qualified stroke center), the alert might include directions to that medical resource. However, if the medical resource is inappropriate for the possible medical condition identified (i.e., the hospital is not qualified to treat strokes, or has an inordinately long wait time) the alert preferably provides location information related to a second medical resource having second qualifications that are better suited for addressing the possible medical condition identified. In the example above, the system 100 might provide directions to another, different hospital that is qualified as a stroke center, even if that location is further away than the first hospital, because the second hospital is likely more appropriate for addressing the possible medical condition identified. In another example, if a closer first ER has a much longer average or actual wait time than a second ER located further away, directions to the second ER might be provided.

Another one of the options (Option C) presented to the user is the option to enter the healthcare system 102 by interacting with the second triage system 112 (Step 220) to further identify and address the possible medical conditions. The second triage system 112 provides a person-to-person communication between the user 104 and a medical provider 120 and/or navigation team that provides guidance and direction to assist the user in navigating the healthcare system 102 and for locating the most appropriate medical treatment for the possible medical condition identified by the system.

Preferably, the medical provider 120 includes one or more telehealth providers that function as healthcare providers, healthcare navigators, and/or medical directors. While each of these roles may be filled by separate provider (i.e., a team of providers working collaboratively), it is more preferable to have medical providers that are cross-trained so that they can function in two or more of these roles. In order to function as a healthcare provider, the medical provider 120 should be trained as a medical doctor, nurse practitioner, or physician assistant with broad-based training and experience, such as emergency medicine, internal medicine, and/or family practice. The medical provider 120 should also be board-certified and be skilled as a diagnostician. The purpose of the medical provider 120, when functioning as a healthcare provider, is to provide healthcare via telehealth to the user including providing prescriptions that are electronically sent to a local pharmacy and any other specialist referrals. Next, the purpose of the medical provider, when functioning as a healthcare navigator, is to provide medical/healthcare navigation to the user. This navigation function might include, for example, guiding possible stroke patients to the nearest stroke center ER instead of just the closest ER. In another example, in the case of a user having a puncture wound, the medical navigator might direct that user to the nearest walk-in clinic that has minimum wait time, depending the severity of the wound. Preferably, the system 100 is provided with global positioning system information and medical facility information, including capabilities, ratings, and wait times, which will enable the medical provider to assist the user in selecting and locating the nearest and most appropriate care option for the potential medical issue. Finally, most states allow first responders (e.g., EMS and fire & rescue personnel) to provide more advanced care in the prehospital stage if directed by an online medical director. As such, the purpose of the medical provider, when functioning as a medical director, is as an online medical director of advanced services, which will enable first responders to provide more advanced care in the prehospital stage.

Preferably, once the user has entered the healthcare system 102, the defined data collected by the system 100 is provided to the medical providers 120 and to the second triage system 112. For example, the defined data may be transferred to a medical provider 120 through text to the telehealth physician and/or connect the physician to the user's electronic medical records, provided the user has authorized sharing of the information.

Since the defined data is collected over a long period of time, rather than simply during a brief medical checkup, and relates to many aspects of the user's life, including several aspects that are generally not considered by medical providers 120, a fuller picture of that user can be obtained after reviewing the defined data. Among other things, making this larger body of defined data available to medical providers 120 will improve patient outcomes and reduce instances of misdiagnoses and mis-utilization of the healthcare system 102. The differential diagnosis generated by the first triage system 110 will also be made available to medical providers 120 for the same reason (i.e., to improve patient outcomes and reduce instances of misdiagnoses and mis-utilization of the healthcare system).

As shown above, the presently-disclosed system 100 is a marriage of modern everyday technology interfacing with a new interpretive technology application to alert the user of potentially unknown health needs that is also paired with follow through per the user's choice to provide definitive care via telehealth or via navigation by a cross trained team of healthcare providers and navigators to the most appropriate healthcare facility or healthcare provider. A major advantage and distinguishing factor of the presently-disclosed system 100, compared to other prior systems, is that the presently-disclosed system is used, initially, by users 104 while they are outside of the healthcare system 102. A further advantage is that, in at least some cases, the system 100 might provide alerts to a user 104 when that user is not even aware that they are potentially in need of medical assistance. For example, this type of unaware user 104 might unknowingly be suffering the early effects of a heart attack or a stroke (e.g., possibly caused by unknown atrial fibrillation). Preferably, the system 100 is configured to alert the user 104 to this potential danger and to give them the option of seeking timely medical care. The system 100 is also intended for use by users 104 after they have entered the healthcare system 102 for medical treatment related to a medical condition. In this latter case, the second triage system 112 functions in a similar fashion as air traffic control in the flight industry. In particular, the second triage system 112 assists users to locate the most appropriate medical facility and medical provider based on the defined data available to the system.

The following examples illustrates use of a hypothetical system 100 named GUARDIAN ANGEL 360™ that implements the systems and methods of the present disclosure. In these hypothetical scenarios, the GUARDIAN ANGEL 360 system is used to assist users in detecting a possible medical condition and then entering and navigating the medical system for diagnosis and treatment of that medical condition.

### EXAMPLE 1: Abnormal Subconscious Behavior Patterns—Passive User Input

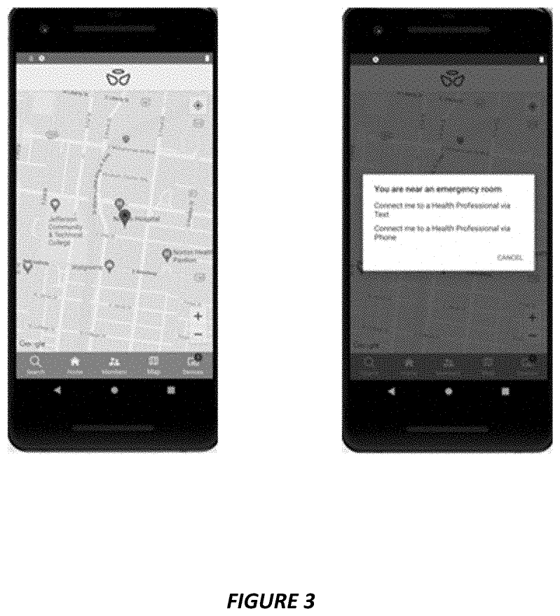

In this first example, the GUARDIAN ANGEL 360 system is used to detect a medical need and provide assistance based on subconscious behavior patterns (i.e., passively-collected/passively-provided defined data) of a user. With reference to FIG. 3, the GUARDIAN ANGEL 360 system uses defined data from the user device (i.e., a mobile phone in this example) and behavior tracing patterns, such as driving times and pattern, diurnal/nocturnal patterns and user activity (e.g., exercise, unusual health data from monitoring device) patterns to detect possible medical needs. Using a mobile phone at an abnormal time for the user (e.g., at 2 AM) or driving toward or within a pre-specified distance from a given point of interest (i.e., geofencing), such as a medical facility or other point of interest would prompt the GUARDIAN ANGEL 360 system to begin building a differential diagnosis. If the GUARDIAN ANGEL 360 system detects a possible medical need, a notification is sent to the user, asking them if they could be having a medical need.

In some embodiments, the GUARDIAN ANGEL 360 system uses at least two methods to identify if a user is navigating to an emergency room. The first method used to determine if a user is navigating to an ER is if the user navigates within a predefined geofenced area close to an ER. In that case, GUARDIAN ANGEL 360 uses the GPS/Navigation function of the mobile device to track when a user enters a predetermined space or distance around an ER (e.g., within a ½ mile radius of the ER or an ER parking area). In certain cases, this space will be defined using one or more of geofencing technology, GPS coordinates, and a radial distance from this defined area. Preferably, the distance from the ER required to trigger a push notification is customizable by the user. When GUARDIAN ANGEL 360 identifies that a user has come within this area, a push notification is issued to the user, which gives the user the option to connect directly with a telehealth provider, such as by text or phone.

Next, GUARDIAN ANGEL 360 might determine that a user is navigating to an ER if they are not following their typical driving patterns, based on other travel data that might be collected by the device 106, and are heading in the direction of an ER. GUARDIAN ANGEL 360 uses predictive analytics and an algorithm to determine if a user is navigating towards an ER and that determination might be made using one or more of the following (or other similar) factors: the user is driving at a non-typical time of day, the user is driving on a non-typical path towards an ER, the user is driving at a typical time of day but on a non-typical path towards an ER, the user has not already selected a destination in their maps app, the user is navigating towards an ER but not at an “emergency” rate of speed. When GUARDIAN ANGEL 360 identifies a combination of these and other factors, it will provide a push notification asking the user if they would like to connect to a telehealth professional. Since the user is likely to be driving an automobile, hands-free options will be available and defaulted to in this scenario.

### EXAMPLE 2: Abnormal Vital Signs—Passive User Input

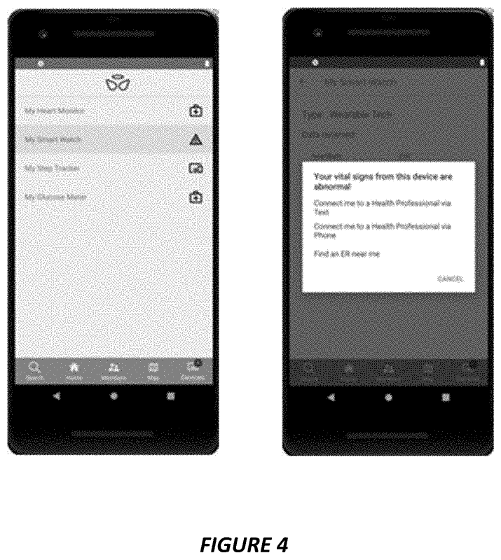

In another example, the GUARDIAN ANGEL 360 system is used to detect a medical need and provide assistance based on abnormal vital signs of a user. For example, with reference to FIG. 4, a heart rate or a sleeping pattern is established by the wearable device. If that heart rate is outside of a normal band of heart rates, such as a slow heart rate that is less than 60 beats per minute, would prompt the GUARDIAN ANGEL 360 system to build a differential diagnosis that might include atrioventricular (AV) nodal heart blocks, sick sinus syndrome, myocardial infraction, hypothyroid, etc. On the other hand, a heart rate greater than 100 would prompt the GUARDIAN ANGEL 360 system to build a different differential diagnosis that might include myocardial infarction, pulmonary embolus, sinus tachycardia, ventricular tachycardia, Supraventricular tachycardia (SVT), anxiety, fever, hyperthyroid, even “silent” gastrointestinal bleeding with anemia for regular rhythms. Alternatively, if the tachycardia was irregular the differential diagnosis could include tachy-brady syndromes, accelerated junctional rhythm, and the sometimes silent and asymptomatic atrial fibrillation with rapid ventricular rate, etc. A similar analysis would detect an irregular sleeping pattern (noise from snoring or heart arrhythmia) and alert through GUARDIAN ANGEL 360 to build the different differential diagnosis.

### EXAMPLE 3: Internet Search Activity—Active User Input

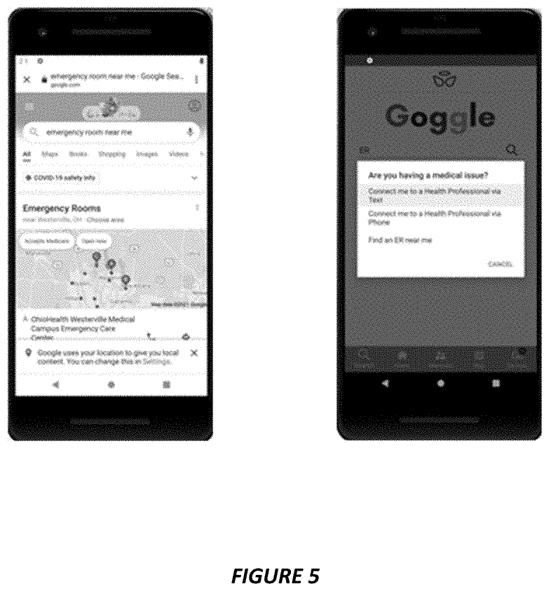

As depicted in FIG. 5, the GUARDIAN ANGEL 360 system may be configured to trigger an alert in response to certain activity taking place on the user's devices (i.e., active user input from the user), such as searches within a mobile phone's search function, Internet searches, etc. For example, an alert might be triggered if the user performs an Internet search for certain terms related to medical facilities, medical conditions, etc. Similarly, if the user searches the maps function of their mobile device for medical facilities, such as an ER, ED, and Emergency Department, an alert might be triggered. In preferred instances, the GUARDIAN ANGEL 360 system will be permitted to read the search engine's API to identify when the user is searching for certain key terms. This is a permission that is given by the user after they have downloaded the GUARDIAN ANGEL 360 application and setup an account. In other cases, the necessary permissions needed for GUARDIAN ANGEL 360 to perform this monitoring function might be granted by the user in an already-installed application on the user's mobile device, such as in the user's health insurance application.

The GUARDIAN ANGEL 360 system builds a differential diagnosis based on the search terms or search activity. For example, searching for an emergency room could generate an emergency differential diagnosis, whereas searching for a dermatologist could generate a dermatologic differential diagnosis. Likewise, if a user searches for medical conditions such as chest pain, the system generates a differential diagnosis for chest pain with cardiac conditions (e.g., myocardial infarction, acute cardiac syndrome, unstable angina or stable angina) and non-cardiac conditions (e.g., pulmonary embolus, costal chondritis, pneumonia, pleurisy, asthma). Further, combined search inquiries, such as a first search related to chest pain combined with a second search related to emergency rooms, could result in the generation of a differential diagnosis where the more serious and emergency conditions are positioned at the top of the of the differential diagnosis.

Once GUARDIAN ANGEL 360 determines that the user is possibly in need of medical assistance, such as when it has identified that a person is searching for an Emergency Room or with certain differential diagnoses, it provides a push notification option to connect them directly to a telehealth physician through text or by phone. In those cases, GUARDIAN ANGEL 360 might further assist the user to find an ER nearby or a medical provider that is best suited to address the user's medical need. For example, GUARDIAN ANGEL 360 might direct a user to the ER having the shortest current wait time or to the ER having capabilities suited for the type of medical issue indicated by the differential diagnosis (e.g., a stroke center when the user is potentially having a stroke).

Additionally, to assist the medical provider selected by the user, GUARDIAN ANGEL 360 is preferably configured to transmit the user's historical medical data plus current monitoring data (e.g., abnormally high heart rates, low 02 saturation, etc.) and electronic medical records to that medical provider to then be linked or associated with the user's Electronic Health Records (EHRs) and Electronic Medical Records (EMRs). Thus, advantageously, the system 100 is preferably configured to provide stored long-term data on patients, where the data is collected includes data collected when that user is not in the healthcare system (e.g., not in the hospital or involved in a home monitoring system). This historical data can assist the medical provider to have a more complete picture of the patient and to make a more accurate diagnosis of that patient's medical condition. For example, suppose a patient with atrial fibrillation is started on a Beta blocker medication to keep his heart rate from going above 100 beats per minute and below 60 beats per minute. GUARDIAN ANGEL 360's long-term collection of the user's daily heart rate would assist a doctor in determining whether to increase or decrease the dosage of the Beta blocker medication or to keep the dosage unchanged. In another instance, the health of a patient with Parkinson's disease might be monitored by GUARDIAN ANGEL 360 by measuring and/or tracking the movement (i.e., tremor) of the patient's thumb on a thumb pad of that patient's mobile device. Providing the patient's doctor with this type of data will assist in titrating their Parkinson's medications. Typically, the doctor would only have the heart rate or tremor measurements taken at the time of the visit to guide therapeutic decisions. Another scenario involves a person's gait changing from their normal gait, where that change is so subtle that the person does not notice the change. The change in gait could indicate that a person with Parkinson's needs reevaluation and medicine adjustments or that a patient with normal pressure hydrocephalus has a ventriculoperitoneal (VP) shunt that needs to be replaced. Thus, GUARDIAN ANGEL 360 provides an early notification of medical conditions that result from subacute behavioral changes to the patient. This early notification will enable doctors to provide early non-emergency intervention and to avoid later emergency intervention and permanent disability.

Although this description contains many specifics, these should not be construed as limiting the scope of the invention but as merely providing illustrations of the presently preferred embodiment thereof, as well as the best mode contemplated by the inventor of carrying out the invention. The invention, as described herein, is susceptible to various modifications and adaptations, as would be understood by those having ordinary skill in the art to which the invention relates. The previous detailed description is of a small number of embodiments for implementing the invention and is not intended to be limiting in scope. The following claims set forth a number of the embodiments of the invention disclosed with greater particularity.

What is claimed is:

1. A pre-medical monitoring and efficient patient navigation system for reducing instances of mis-utilization of medical resources and for efficiently navigating a user to medical resources within a healthcare system that are most appropriate for providing medical care required by the user, the system comprising:
one or more technology devices, each with at least one data input receiver for receiving defined data pertaining to the user that is collected prior to the user entering the healthcare system;
a data repository for receiving and storing the defined data from the one or more technology devices;
a first triage system located outside of the healthcare system and comprising:
a differential diagnosis logic configured to automatically receive the defined data from the data repository and to then automatically analyze the defined data to identify possible medical conditions that might be affecting the user based on the defined data received; and
an alert generator for issuing an alert to at least one of the one or more technology devices for review by the user in response to an alert trigger that is based on the analysis performed by the differential diagnosis logic;
a second triage system located within the healthcare system and comprising a call center having navigation personnel and facilities for communicating with the user, wherein the one or more options to enter the healthcare system provided by the first triage system includes an option to contact the call center and to initiate a person-to-person conversation with the navigation personnel.

2. The system of claim 1 wherein the alert trigger comprises the differential diagnosis logic identifying a possible medical condition, wherein the alert includes information related to the possible medical condition and one or more options to enter the healthcare system for the purpose of obtaining medical treatment for the possible medical condition.

3. The system of claim 1 wherein the alert trigger comprises the differential diagnosis logic identifying that the one of the one or more technology devices is located within a specified proximity of or is navigating towards a first medical resource having first qualifications.

4. The system of claim 3 wherein, when the differential diagnosis logic identifies a possible medical condition impacting the user, the alert provides location information related to a second medical resource having second qualifications either through the triage 1 or triage 2 states as defined above, where the second qualifications are better suited for addressing the possible medical condition identified by the differential diagnosis logic than the first qualifications of the first medical resource.
